(19) United States
(12) Patent Application Publication
(10) Pub. No.: US 2026/0047887 A1
(43) Pub. Date: Feb. 19, 2026

(54) Modeling Tools for Total Shoulder Arthroplasty Pre-Operative Planning

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Alessandro Navacchia, Broomfield, CO (US); Elizabeth Duxbury, Kernville, CA (US); Nathan A. Netravali, Littleton, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/103,914

(22) PCT Filed: Sep. 7, 2023
(86) PCT No.: PCT/US2023/032212

§ 371 Date: Feb. 14, 2025

Related U.S. Application Data
(60) Provisional application No. 63/405,223, filed on Sep. 9, 2022.

(51) Int. Cl. A61B 34/10; A61F 2/46; G16H 20/40

(52) U.S. Cl. CPC A61B 34/10; A61F 2/4612; G16H 20/40; A61B 2034/104; A61B 2034/105; A61B 2034/108; A61F 2002/4633

(57) ABSTRACT

Disclosed herein is a system and method for performing pre-operative planning of total joint arthroplasty. The planning tool builds a model and analyzes and visualizes movement of the joint for various selections of implant models and placement of the components on the implants on the patient's anatomy. The tool also analyzes and visualizes motions of the joint during common activities of daily living and analyzes and visualizes changes in muscles.

### RELATED APPLICATIONS

The application claims the benefit of U.S. Provisional Patent Application Ser. No. 63/405,223, entitled “Modeling Tools for Total Shoulder Arthroplasty Pre-Operative Planning”, filed Sep. 9, 2022, the contents of which are incorporated herein in their entirety.

### FIELD

The present disclosure relates generally to orthopedic procedures, for example, total joint replacement procedures, and, in particular, to total shoulder arthroplasty (TSA) procedures.

### BACKGROUND

Orthopedic procedures to replace joints, for example, shoulders, are well known and commonplace in today's society. Total shoulder replacement, also known as total shoulder arthroplasty (TSA), is the removal of portions of the shoulder joint, which are replaced with artificial implants to reduce pain and restore range of rotation and mobility. In an anatomic TSA procedure, which may be used, for example, when osteoarthritis has compromised portions of the shoulder joint, the ball portion of the shoulder joint is attached to the top of the humerus and the cup portion of the shoulder joint is attached to the glenoid. In a reverse TSA procedure, which may be used, for example, when the rotator cuff has been compromised, the ball portion of the shoulder joint is placed on the glenoid while the cup portion of the shoulder joint is attached to the top of the humerus.

Current state-of-the-art planning for TSA consists of using a computed tomography (CT) scan from the patient to correctly size and position implant components on the patient's specific anatomy. One of the objectives of TSA is to restore normal shoulder function, which includes both mobility and stability. Pre-operative prediction of the implanted joint range-of-motion (RoM) can facilitate optimization of post-operative mobility. Collision between the humeral liner and the scapula due to limited RoM in adduction has been identified as a cause of scapular notching, which has been reported to occur in more than half of reverse TSA patients and has been related to glenoid loosening and implant failure. In addition, limited RoM and impingement can lead to implant instability and dislocation, which is one of the leading causes for revision surgery. Reverse TSA jump distance calculation can provide insight into the trade-off between mobility and stability of the joint.

In addition, post-operative mobility and stability of the joint are significantly impacted by muscle function. Although this is true for every joint replacement, this is especially relevant to TSA as joint stability is primarily provided by muscle compressive forces and implant design (e.g., anatomic vs reverse) has a significant impact on muscle moment arms and muscle force generation capacity. Therefore, pre-operative knowledge of patient-specific post-operative muscle function can further improve the surgeon's ability to optimally select implant design, size, and placement.

Muscle forces are the main determinant of contact mechanics. Large rotational moments at the implant-bone interface due to the joint contact force line of action can lead to glenoid component loosening (e.g., “rocking-horse” failure mechanism for anatomic TSA). Therefore, prediction of contact mechanics during activities of daily living can provide valuable insight into the risk of failure of the implant.

Several commercially-available planning applications provide the ability to calculate shoulder impingement-free RoM in its primary degrees of freedom (i.e., flexion/extension, abduction/adduction, internal/external rotation) by rotating the implanted humerus about the three axes of rotation and detecting at what rotation bone-on-bone or implant-on-bone impingements occur. However, joint RoM in its main degrees of freedom is hard to link to the mobility needs of a patient during daily life. Therefore, although TSA is supposed to restore joint mobility, the knowledge of the specific mobility patterns required by a patient to perform activities of daily living would better inform surgery planning.

Generic knowledge of how implant design (anatomic vs reverse) affects post-operative muscle function is available in the scientific literature. However, it is difficult to extrapolate such generic knowledge to a specific patient, given the broad spectrum of osteoarthritic joint deformity and pre-operative muscle state. Currently available planning tools do not incorporate any prediction of post-operative muscle function nor do they predict the post-operative RoM or the ability of the patient to perform activities of daily living post-operatively.

### SUMMARY

Disclosed herein is a system and method for providing surgical pre-planning of both anatomic and reverse TSA procedures. 3D modeling tools are used together with CT-based patient anatomy within TSA planning software to simulate post-operative shoulder RoM in the joint's primary degrees of freedom to provide insight into the TSA mobility-stability trade-off on a patient-specific basis. Additionally, the present disclosure provides simulation of post-operative glenohumeral motion during activities of daily living relevant to normal shoulder function to inform implant selection and placement with real life mobility patterns. The present disclosure also simulates post-operative muscle (i.e., deltoid, rotator cuff) function (i.e., moment arm, length, force generating capacity) to inform implant selection and placement. Lastly, the present disclosure performs muscle-driven simulations of activities of daily living to predict implant contact mechanics and estimate risk of glenoid component loosening.

In a first example, a method for performing pre-operative planning of joint replacement surgery comprises deriving a model of the anatomy of a joint to be replaced from images gathered from one or more imaging modalities, receiving a selection of an implant and placement of the implant on the anatomy of the joint and analyzing and visualizing, on the model of the joint, a range-of-motion of the joint for the selected implant and placement of the implant.

In any preceding or subsequent example, the method further comprises visualizing the placement of the implant on the model of the joint.

In any preceding or subsequent example, the method further comprises providing an animated visualization of the model through a range-of-motion for each major axis of rotation.

In any preceding or subsequent example, the method further comprises wherein the animated visualization automatically moves the model through the ranges-of-motion.

In any preceding or subsequent example, the method further comprises wherein the animated visualization is controlled using a manual input of the ranges-of-motion.

In any preceding or subsequent example, the method further comprises providing a graphical visualization of the range-of-motion with respect to each of the three axes of motion.

In any preceding or subsequent example, the method further comprises wherein the graphic visualization of the ranges-of-motion is a graph.

In any preceding or subsequent example, the method further comprises wherein the graphic visualization of the ranges-of-motion is an animated skeletal representation of the joint.

In any preceding or subsequent example, the method further comprises providing one or more views of the imagery from which the model was derived showing placement of components of the implant thereon.

In any preceding or subsequent example, the method further comprises providing an animated visualization of the ranges-of-motion of the model required for performance of one or more activities of daily living.

In any preceding or subsequent example, the method further comprises informing the user of parts of the bone and/or implant that are impinging or colliding during range-of-motion visualizations.

In any preceding or subsequent example, the method further comprises highlighting parts of the patient's anatomy where impingement occurs.

In any preceding or subsequent example, the method further comprises providing a graphical visualization of the ranges-of-motion with respect to each of the three axes of rotation for the one or more activities of daily living.

In any preceding or subsequent example, the method further comprises providing a selection mechanism for selecting the activity of daily living to be animated.

In any preceding or subsequent example, the method further comprises providing a visualization of the change in a location of a center of rotation of the joint on a visualization of the model.

In any preceding or subsequent example, the method further comprises wherein the visualization of the model comprises one or more muscles visualized on the model.

In any preceding or subsequent example, the method further comprises providing a selection mechanism for selecting the muscles to be visualized on the model.

In any preceding or subsequent example, the method further comprises providing a graphical visualization of the moment arm of the one or more muscles selected with the selection mechanism.

In any preceding or subsequent example, the method further comprises providing a graphical visualization of the force-length curve through relevant ranges-of-motion for the one or more muscles selected with the selection mechanism.

In any preceding or subsequent example, the method further comprises receiving a selection of one or more additional implant models and placement of the models on the patient's anatomy.

In any preceding or subsequent example, the method further comprises providing a comparison between the originally-selected implant model and the one or more additional selected implant models.

In any preceding or subsequent example, the method further comprises providing a graphical visualization of the range-of-motion with respect to each of the three axes of rotation for each of the selected implant models.

In any preceding or subsequent example, the method further comprises suggesting implant positions that optimize certain parameters such as muscle moment arm, force-length curve, or minimal impingement.

In another example, a system for performing pre-operative planning of joint replacement surgery comprises a processor and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, providing an application for analyzing and visualizing on the model a range-of-motion of the joint for a particular implant and placement of components of the implant on the anatomy of the joint.

In any preceding or subsequent example, the software causes the system to perform the further function of providing an application for analyzing and visualizing a range-of-motion of the joint required for performance of one or more activities of daily living.

In any preceding or subsequent example, the software causes the system to perform the further function of providing application for analyzing and visualizing a change in a location of a center of rotation of the joint in a visualization of the model, wherein the visualization of the model comprises one or more muscles displayed on the model.

In any preceding or subsequent example, the system further comprises wherein the processor is a surgical computer provided as part of a computer-assisted surgical system.

In another example, a system for performing pre-operative planning of joint replacement surgery comprises a processor and software that, when executed on the processor, causes the system to perform the functions of providing a workflow, the workflow accepting as input patient-specific imaging of anatomy of a joint to be replaced using an implant from one or more imaging modalities, deriving, from the one or more imaging modalities, a model of the joint, providing an application for analyzing and visualizing on the model, a location of a center of rotation of the joint in a visualization of the model, wherein the visualization of the model comprises one or more muscles displayed on the model.

Examples of the present disclosure provide numerous advantages. For example, pre-operative planning performance is improved via the enhanced prediction of post-operative mobility and stability. Additionally, the disclosed system and method provide more personalized planning which can lead to clinical benefits such as improved patient function and reduced risk of dislocation or failure. The disclosed system and method further enhance the ability of the surgeon to interpret several patient-specific biomechanical quantities.

Further features and advantages of at least some of the examples disclosed herein, as well as the structure and operation of various examples, are described in detail below with reference to the accompanying drawings.

### BRIEF DESCRIPTION OF THE DRAWINGS

By way of example, specific examples of the disclosed system and method will now be described, with reference to the accompanying drawings, in which:

FIG. 1 is a block diagram showing an exemplary workflow in accordance with one or more features of the present disclosure.

FIG. 2 shows an exemplary user interface screen from the range-of-motion analysis tool.

FIG. 3 shows an exemplary user interface screen from the activities of daily living analysis tool.

FIG. 4 shows an exemplary user interface screen from the muscle analysis tool.

FIG. 5 shows an exemplary user interface screen from the comparison tool.

FIG. 6 depicts an operating theatre including an illustrative computer-assisted surgical system with which various examples of the disclosed system and method could be used.

### DEFINITIONS

For the purposes of this disclosure, the term “implant” is used to refer to a permanent or temporary prosthetic device or structure manufactured to replace or enhance a biological structure.

Although much of this disclosure refers to surgeons or other medical professionals by specific job title or role, nothing in this disclosure is intended to be limited to a specific job title or function. As such, the term “user”, as used herein should be interpreted to include any doctor, nurse, medical professional, or technician, or any other user of the system or practitioner of the method. Any of these terms or job titles can be used interchangeably with the user of the systems disclosed herein unless otherwise explicitly stated.

### DETAILED DESCRIPTION

The system and method disclosed herein provides a tool for pre-operative planning of a joint replacement surgery which will assist the user in selecting the proper implant and planning and executing the surgery such as to minimize both intra-operative and post-operative negative outcomes for the patient. Note that, although the disclosed system and method are explained in terms of a total shoulder replacement procedure, all or portions of the disclosed system and method may be modified such as to be applicable to any joint replacement procedure.

In a TSA procedure, the different approaches to the surgery provide different outcomes. For example, in an anatomic TSA procedure, the result provides in most cases similar range-of-motion and similar biomechanics to the user's native anatomy. A reverse TSA procedure, on the other hand, tends to provide reduced range-of-motion and biomechanics that have been altered from the user's native anatomy. For example, in the reverse procedure, the range-of-motion may be limited by bone-to-bone, implant-to-implant, or implant-to-bone impingement, which may lead to instability of the joint. Additionally, the reverse procedure may result in a medialized center of rotation and humerus distalization, which results in changes in the length, moment arms and function of the various muscles of the joint.

The present disclosure provides the user with a means for predicting and evaluating post-operative results given the variables of the type of procedure to be used, the specific implant selected, the particular placement of the implant on the patient's anatomy and the changes to the patient's anatomy resulting from the selection and placement of a particular implant, both for the anatomical procedure and the reverse procedure. The present disclosure provides a tool for analyzing and visualizing a predicted range-of-motion (RoM), given various selections of implants and placements thereof, a tool for analyzing and visualizing a predicted ability of the patient to perform various activities of daily living and a tool for analyzing and visualizing predicted changes to the patient's anatomy, namely, the changes to the center of rotation of the joint and the moment arm and length of the muscles. In certain examples, the system and method may provide recommendations for optimized outcomes based on preferences of the surgeon and/or patient.

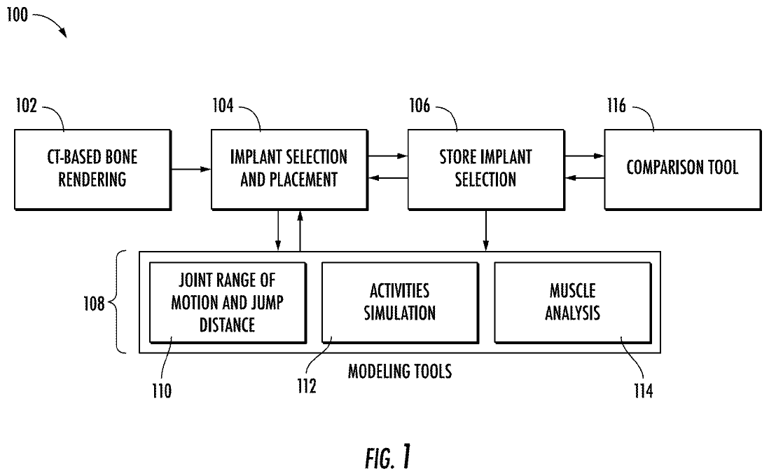

FIG. 1 is a block diagram showing an exemplary workflow 100 in accordance with one or more features of the present disclosure. The present disclosure relies on the creation of a model of the anatomy of the specific patient showing the bone structures that will be affected by placement of the implant. In certain examples, at step 102 of workflow 100, the model may be derived from a CT scan. In other examples, the model may be derived using one or more other imaging modalities, including, for example, MRI, X-ray or ultrasound, or various combinations thereof. For example, a CT scan may show the bones of the joint while an MRI can show soft tissue structures in addition to bones. In certain examples, the model can also be derived using an atlas or statistical shape model based on information gathered intraoperatively using anatomical landmarks gathered during procedures.

At step 104 of workflow 100, the surgeon specifies to the tool the type of surgery to be performed, a selection of a particular model of implant (including various characteristics of the implant, for example, size of the various components) and a specific placement of the implant on the patient's anatomy. In certain examples, workflow 100 can also suggest an initial placement of the implant based on the anatomy of the patient or on a simulation.

At step 106 of workflow 100, the specific implant selection and placement from step 102 will be stored for use by the analysis tools. At this point in workflow 100, the user can access the suite 108 of analysis and visualization tools which include, in some examples, a tool providing a range-of-motion analysis 110, a tool for simulating activities of daily living 112 and a muscle analysis tool 114, based on the model of the patient's anatomy and the specific implant selection and placement information provided in step 104 of workflow 100. The analysis tools in suite 108 will be explained in more detail below. At any time during the use of the analysis and visualization tools in suite 108, the user may change the implant selection and placement information.

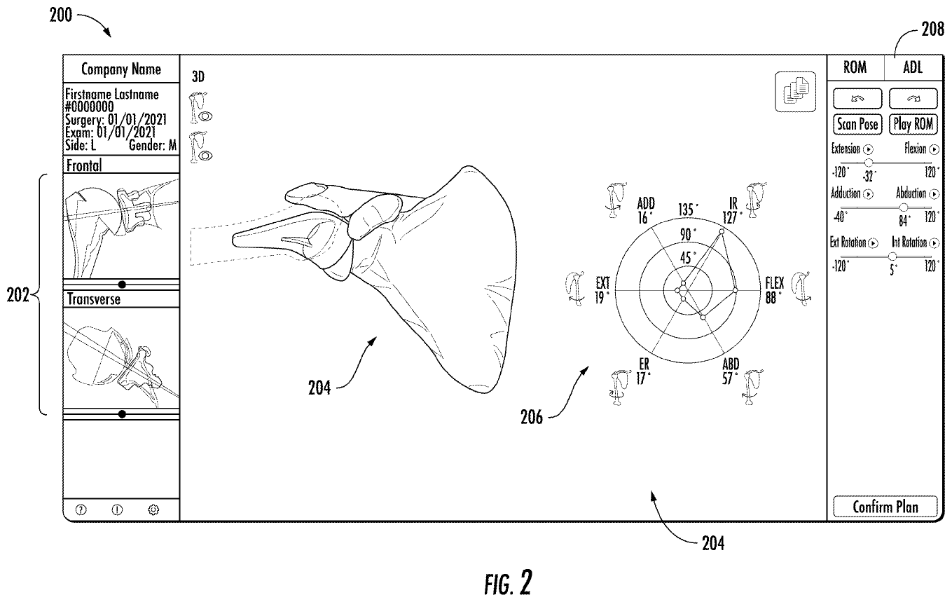

An exemplary screen 200 of the range-of-motion analysis and visualization tool 110 is shown in FIG. 2. As would be realized by one of skill in the art, the arrangement of information on the screen and the manner of expressing the information may be changed without departing from the spirit or scope of the present disclosure.

In one example, user interface 200 shows one or more views 202 from the imagery used to generate the model of the patient's bone anatomy. The one or more views 202 may show the placement of the various components of the implant overlaid on the imagery. In the specific case shown in FIG. 2, the implant components for a reverse TSA procedure are shown.

Exemplary user interface 200 may also show a model 204 of the joint which has been derived from the imagery collected in step 102 of workflow 100. The model 204 shows the specified positioning of the components of the selected implant. In certain examples, the model may be animated to show a kinematic simulation of glenohumeral motion. The model of the joint 204 may be placed in various positions using manual controls shown in area 208 of user interface 200. In some examples, the manual control may be sliders, as shown in FIG. 2. Alternatively, or in addition to, the tool may automatically move the model of the joint 204 through a full range of possible motions, which are visualized on an animation of model 204.

The method calculates impingement-free ranges-of-motion in the shoulder's main degrees of freedom (i.e., abduction/abduction, flexion/extension, interior rotation/exterior rotation) for the current implant selection and placement chosen by the user. Each time the user changes the implant or placement, the software calculates the joint ranges-of-motion by sequentially rotating the bone and implant geometries about the three main axis of rotation and detects bone-on-bone, implant-on-implant, and/or implant-on-bone impingement using a collision detection algorithm. In some examples, the user may be informed of the parts of the patient's anatomy wherein impingement which limits the range-of-motion occurs, for example, by highlights those parts of the anatomy.

A visualization 206 of the joint ranges-of-motion in the primary degrees of freedom is presented, with the polygon denoting the available range-of-motion without impingement. As would be realized by one of skill in the art, many other methods of denoting the available range-of-motion without impingement could be used. An additional indicator of the available range-of-motion is shown in area 208 of user interface 200 which shows the colored portions of the sliders to indicate the available range-of-motion while the gray areas of the sliders indicate areas of impingement. The slider bars in area 208 of user interface 200 may also be used to manually control the joint angles shown in animated model 204.

For reverse TSA procedures, calculation of and a visualization of the jump distance may be presented on this or on a different screen (not shown). The jump distance is the distance that the glenosphere needs to travel to dislocate from the liner, after impingement in the primary degrees of freedom. The jump distance is a quantification of joint conformity and stability provided purely by the geometry of the components. Because implant performance is a trade-off between mobility and stability, it is important to evaluate both the ranges-of-motion and the jump distance simultaneously when selecting implant parameters and placement.

The user may change the selection of or placement of the components of the implant at any time. This may be accomplished by returning to step 104 or workflow 100. Within user interface 200, this may be accomplished by clicking on the one or more views 202 of the native imagery. Alternatively, controls (not shown) may be provided directly on screen 202 to affect a change in the selection or placement of the implant.

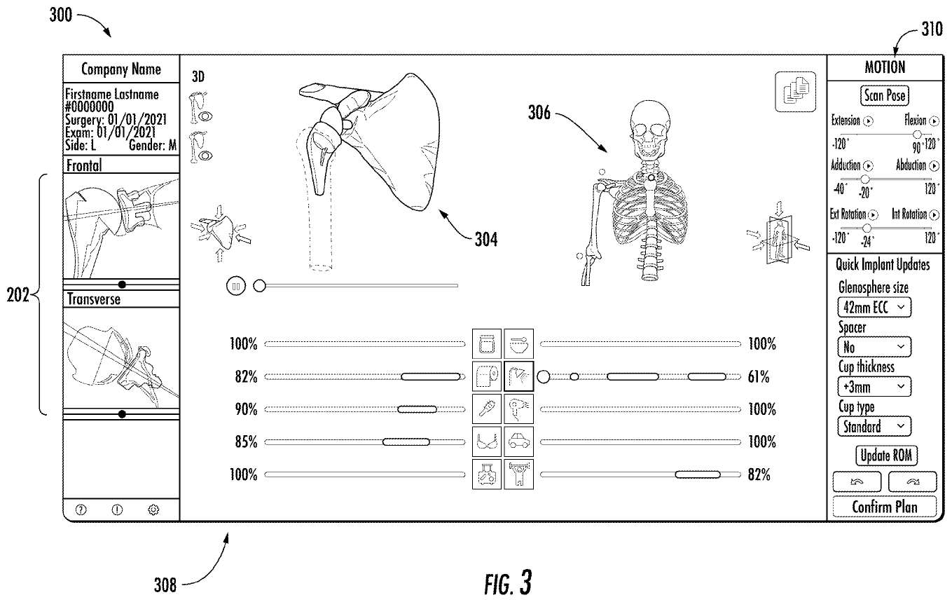

Activities simulation tool 112 provides a kinematic simulation of typical glenohumeral motion that occurs during activities of daily living. An analysis and visualization of the joint when moved within ranges-of-motion associated with various activities of daily living relevant to shoulder function, for example, reaching behind back, drinking from a cup, reaching the contralateral shoulder, etc. is provided in user interface screen 300 shown in FIG. 3. Sport-specific motions (e.g., a golf or tennis swing, etc.) can also be considered.

As with the user interface screen 200, exemplary user interface screen 300 shows one or more views 202 from the imagery used to generate the model of the patient's bone anatomy, model and placement of the implant shown thereon. The exemplary user interface screen shown in FIG. 3 also shows a reverse TSA procedure.

In some examples, generic shoulder kinematics are calculated from motion capture data of relevant activities and stored within the tool. In other examples, the kinematics can be derived from other sensors, for example, wearable sensors, fluoroscopy, x-ray, etc. Patient-specific shoulder kinematics can also be measured from the patient's contralateral joint, if healthy, and imported to the software to provide better patient-specific insight. Given an implant selection and placement, the user can select an activity of interest from a list of implemented activities 306. User interface screen 300 includes a model 304 similar to model 204 shown in FIG. 2. After the desired activity of daily living is selected from the list in 308, model 304 is animated to show the kinematic simulation during the selected activity of daily living. For example, the exemplary user interface screen 300 shows the showering activity highlighted within list 308. As such, the kinematic simulation of model 304 shows the ranges-of-motion required during a typical showering activity.

FIG. 3 shows a second method 306 of visualizing the ranges-of-motion. In this case, an animated skeleton 306 is shown with the shoulder joint animated to show the motions of the joint during the selected activity of daily living. As would be realized by one of skill in the art, the visualization 206 of the range-of-motion for the range-of-motion analysis and visualization tool shown in the user interface screen 200 of FIG. 2 may be used on this screen as well and, conversely, the visualization 306 for the activities analysis of visualization tool shown in FIG. 3. may be used as a visualization for the range-of-motion tool 110. Other methods of visualizing the range-of-motion are contemplated be within the scope of the present disclosure.

Potential bone-to-bone, implant-to-implant and/or implant-to-bone impingement is detected using the same collision detection algorithm and will be displayed within the 3D visualization of the patient's shoulder 308. In addition, the ranges-of-motion for each of the activities of daily living for each of the main axes is also shown in area 310 of user interface screen 300. In user interface screen 300, the controls in area 310 may be manipulated to manually show the position of the bones of the joint in model 304 or the range-of-motion in visualization 306. As with the user interface screen 200 of FIG. 2, the colored portions of the sliders indicate available ranges-of-motion, while the grayed-out areas of the sliders indicate areas of impingement. In other examples, other types of controls may be used and may show the ranges-of-motion in other ways and are contemplated to be within the scope of the present disclosure. The list of typical activities of daily living 308 also includes a visualization of the percentage of the activity cycle that cannot be performed without impingement, wherein the colored areas in the bars next to each icon for each activity of daily living show potential restrictions of the range-of-motion wherein the colors of the areas may be varied to show the severity of the restriction.

Suite 108 includes muscle analysis tool 114. As previously discussed, during the reverse TSA procedure, the center of rotation of the joint may change which may cause the moment arm and/or path of the muscles to change. In addition, the muscles may be required to lengthen or shorten depending upon the change in the path. In some examples, muscle analysis tool 114 provides muscle-driven simulations of the joint to provide an estimation of realistic implant contact mechanics, which can be used to calculate mechanical quantities related to risk of implant failure such as rotational moment at the implant-bone interface, implant micromotion, and implant wear. In other examples, the muscle analysis tool 114 could provide other analyses, for example, moment arm calculations throughout the joint range-of-motion or throughout activity cycle, force-generating capacity throughout the joint range-of-motion or throughout activity cycle, etc.

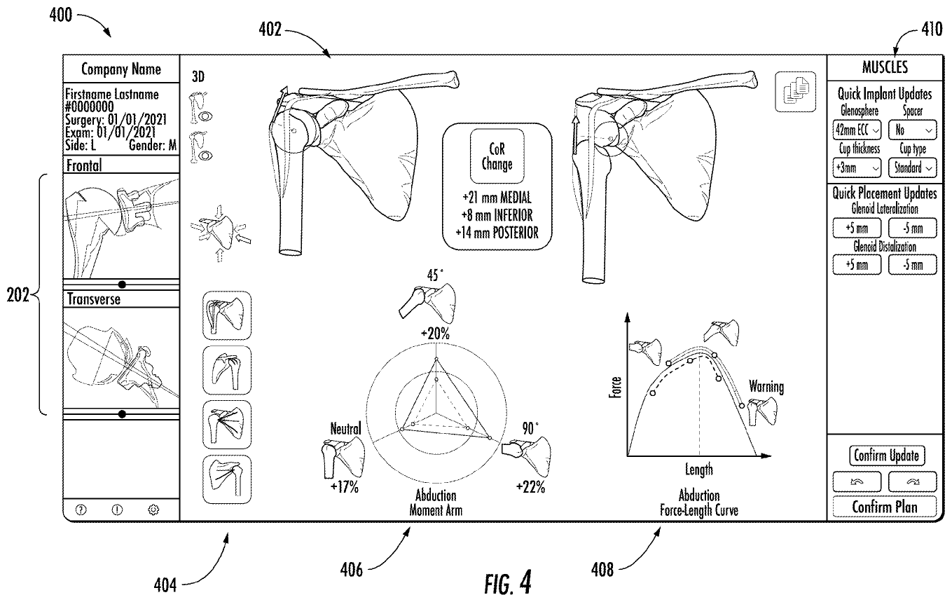

FIG. 4 shows exemplary user interface screen 400 for the muscle analysis tool 114. Model 402 shows various views of a selected muscle on the model and the change in the center of rotation of the joint from the native anatomy of the patient. The particular muscle displayed on model 402 may be selected using one of buttons 404 which, when selected, changes the muscle shown in model 402. A change in the center of rotation of the joint will also cause a change in the moment arm of various muscles of the joint (i.e., the distance between the muscle and the center of rotation of the joint), which is indicative of the ability of the muscle to generate a particular motion. The greater the moment arm of the muscle the easier it is for the muscle to manipulate the bones. Area 406 of user interface screen 400 visualizes the change in the moment arm, from the natural state to a predicted state post-surgery. User interface screen 400 also may provide a force-length curve 408 which provides a comparison of the length of the muscle in its natural state as opposed to the predicted post-operative length. A selection of any one of the buttons in list 404 will change each of model 402, showing the change in the center of rotation and display 406 showing the change in the moment arm and force-length curve 408 to reflect visualization of the muscle that has been selected using button 404. User interface screen 400 also includes area 410 which shows an exemplary interface for changing the positioning of the components of the implant.

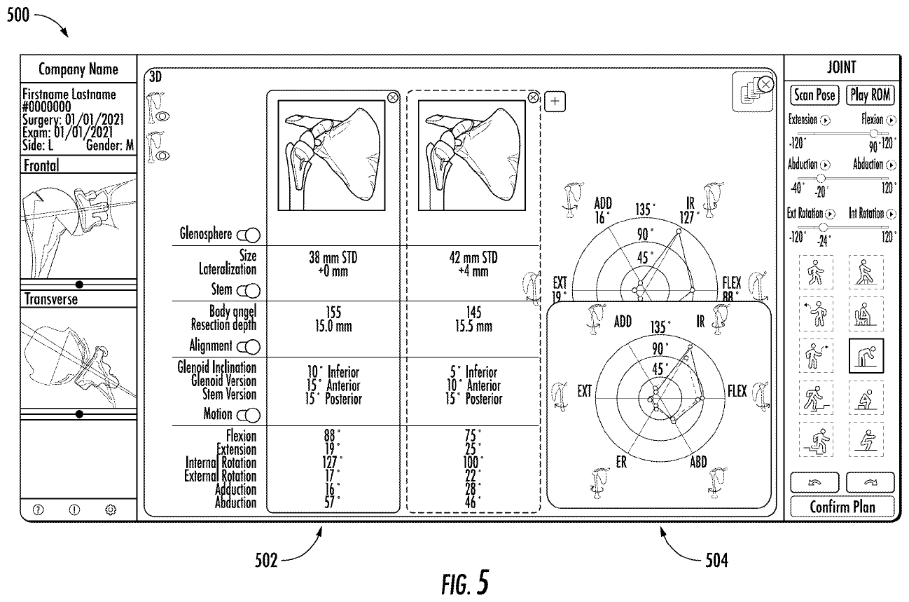

In various examples of the present disclosure, modeling tools 110, 112 and 114 in suite 108 can be accessed by the user at any time as long as an implant is fully selected and positioned. Alternatively, the user can store an implant selection at step 106 of workflow 100 within a list of comparison cases and the modeling tools will present an intuitive comparison of the calculated biomechanical quantities between selected cases. A user interface screen 500 for the comparison tool 116 is shown in FIG. 5. Comparison tool 116 allows a side-by-side comparison 502 of various characteristics of the joint resulting when implants and the positioning of their components is varied. Various aspects of the joint may also be shown. For example, a visualization 504 comparing the ranges-of-motion provided by either of the selected implants may be provided, with the ranges-of-motion for the different implant shown in different colors within visualization 504.

In various examples, any or all of tools 110, 112 and 114 may or may not be used by the user. For example, the muscle analysis tool 114 is most useful for reverse TSA procedures but may not useful when an anatomical TSA procedure undertaken.

In certain examples of the present disclosure, modeling and simulation results from a number of patient-specific cases may be used to generate artificial intelligence or machine learning based implant selection and placement clusters. Such clusters may be representative of the surgeons planning philosophies or techniques, which may be used to provide initial placement and selection within the planning tool that better align with the surgeon's preferences or the patient's preferences or with other post-operative measures. In certain other examples, the system may suggest implant positions that optimize certain parameters such as muscle moment arm, force-length curve, or minimal impingement.

The modeling tools 110, 112 and 114, the comparison tool 116 as well as the user interface allowing the user to select and specify the implant and the placement of the components of the implant may be implemented as one or more applications executing on a system comprising a processor and memory, storing software that, when executed by the processor, performs the functions of the one or more applications comprising the method.

The intra-operative workflow 200 may be implemented as software application running on a computing system interfaced with a navigated or robotic-assisted surgical platform or, alternatively, as part of the software of the navigated or robotic-assisted surgical platform.

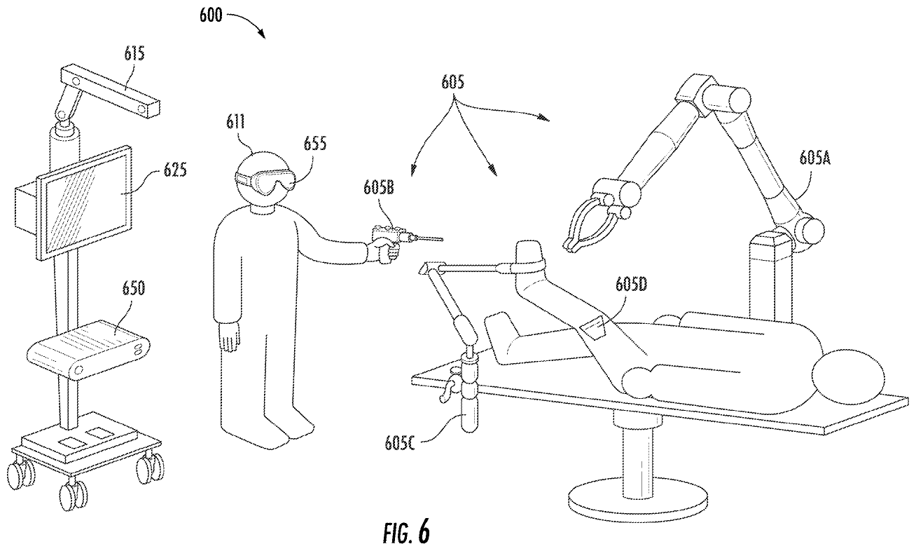

FIG. 6 provides an illustration of an exemplary computer-assisted surgical system 600, with which the system and method of the present disclosure may be implemented. System 600 uses computers, robotics, and imaging technology to aid surgeons in performing orthopedic surgery procedures such as total shoulder arthroplasty. For example, surgical navigation systems can aid surgeons in locating patient anatomical structures, guiding surgical instruments, and implanting medical devices with a high degree of accuracy. Surgical navigation systems 600 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 605 positions surgical tools relative to a patient during surgery. The exact components of the effector platform 605 will vary, depending on the example employed. For example, for a knee surgery, the effector platform 605 may include an end effector 605B that holds surgical tools or instruments during their use. The end effector 605B may be a handheld device or instrument used by the surgeon (e.g., a NAVIO® or CORI® hand piece or a cutting guide or jig) or, alternatively, the end effector 605B can include a device or instrument held or positioned by a robotic arm 605A.

The effector platform 605 can include a limb positioner 605C for positioning the patient's limbs during surgery. One example of a limb positioner 605C is the SMITH & NEPHEW SPIDER2 system. The limb positioner 605C may be operated manually by the surgeon or alternatively change limb positions based on instructions received from the surgical computer 650 (described below).

Resection equipment (e.g., end effector 605B) in FIG. 6) performs bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, and laser ablation systems. In some examples, the resection equipment is held and operated by the surgeon during surgery. In other examples, the effector platform 605 may be used to hold the resection equipment during use.

The effector platform 605 can also include a cutting guide or jig 605D that is used to guide saws or drills used to resect tissue during surgery. Such cutting guides 605D can be formed integrally as part of the effector platform 605 or robotic arm 605A or cutting guides can be separate structures that can be removably attached to the effector platform 605 or robotic arm 605A. The effector platform 605 or robotic arm 605A can be controlled by the system 600 to position a cutting guide or jig 605D adjacent to the patient's anatomy in accordance with a pre-operatively or intra-operatively developed surgical plan such that the cutting guide or jig will produce a precise bone cut in accordance with the surgical plan.

The tracking system 615 uses one or more sensors to collect real-time position data that locates the patient's anatomy and surgical instruments. For example, for TKA procedures, the tracking system 615 may provide a location and orientation of the end effector 605B during the procedure. In addition to positional data, data from the tracking system 615 can also be used to infer velocity/acceleration of anatomy/instrumentation, which can be used for tool control. In some examples, the tracking system 615 may use a tracker array attached to the end effector 605B to determine the location and orientation of the end effector 605B. The position of the end effector 605B may be inferred based on the position and orientation of the tracking system 615 and a known relationship in three-dimensional space between the tracking system 615 and the end effector 605B. Various types of tracking systems may be used in various examples of the present disclosure including, without limitation, Infrared (IR) tracking systems, electromagnetic (EM) tracking systems, video or image based tracking systems, and ultrasound registration and tracking systems.

The display 625 provides graphical user interfaces (GUIs) that display images and the user interface of the navigated surgical platform, as well as the user interface of the systems and methods of the present disclosure, shown in FIGS. 2-5, as well other information relevant to the surgery. For example, in some examples, the display 625 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 625 may include, for example, one or more computer monitors. As an alternative or supplement to the display 625, one or more members of the surgical staff may wear an augmented reality (AR) Head Mounted Device (HMD). For example, in FIG. 6 the surgeon 611 is wearing an AR HMD 655 that may, for example, overlay pre-operative image data on the patient or provide surgical planning suggestions. Various example uses of the AR HMD 655 in surgical procedures are detailed in the sections that follow.

Surgical computer 650 provides control instructions to various components of system 100, collects data from those components, and provides general processing for various data needed during surgery. In some examples, the surgical computer 650 is a general purpose computer. In other examples, the surgical computer 650 may be a parallel computing platform that uses multiple central processing units (CPUs) or graphics processing units (GPU) to perform processing. In some examples, the surgical computer 650 is connected to a remote server over one or more computer networks (e.g., the Internet). The remote server can be used, for example, for storage of data or execution of computationally intensive processing tasks.

In various examples, the one or more applications 110, 112, 114 and 116, as well as workflow 100 of the present disclosure may be implemented as one or more software applications executing on surgical computer 650 integrated with or separate from the software controlling the navigated surgical platform. Also, in various examples, the pre-operative workflow 100 may be implemented as a software application executing on a computing platform other than surgical computer 650, such as a laptop or tablet computing device. The computing device executing workflow 100 may be in communication with surgical computer 650 such as to provide the ability to transfer the results of the analyses to surgical computer 650.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

The present disclosure has been described in the context of specific examples, which are intended only as examples of the disclosure. As would be realized, many variations of the described examples are possible. For example, variations in the design, shape, size, location, function and operation of various components, including both software and hardware components, would still be considered to be within the scope of the disclosure, which is defined by the following claims.

1. A method for performing pre-operative planning of joint replacement surgery comprising:
deriving a model of the anatomy of a joint to be replaced from images gathered from one or more imaging modalities;
receiving a selection of an implant and placement of the implant on the anatomy of the joint; and
analyzing and visualizing, on the model of the joint, a range-of-motion of the joint for the selected implant and placement of the implant.

2. The method of claim 1, further comprising:
visualizing the placement of the implant on the model of the joint.

3. The method of claim 1. further comprising:
providing an animated visualization of the model through a range-of-motion for each major axis of rotation;
wherein:
the animated visualization automatically moves the model through the ranges-of-motion; or
the animated visualization is controlled using a manual input of the ranges-of-motion.

4. (canceled)

5. (canceled)

6. The method of claim 1, further comprising:
providing a graphical visualization of the range-of-motion with respect to each of the three axes of motion.

7. The method of claim 6, wherein the graphic visualization of the ranges-of-motion is one of a graph and an animated skeletal representation of the joint.

8. (canceled)

9. The method of claim 1, further comprising:
providing one or more views of the imagery from which the model was derived showing placement of components of the implant thereon.

10. The method of claim 1, further comprising:
providing an animated visualization of the ranges-of-motion of the model required for performance of one or more activities of daily living.

11. The method of claim 1, further comprising:
informing the user of parts of the bone and/or the implant that are impinging or colliding during range-of-motion visualizations.

12. The method of claim 11, further comprising:
highlighting parts of the patient's anatomy where impingement occurs.

13. The method of claim 1, further comprising:
providing a graphical visualization of the ranges-of-motion with respect to each of the three axes of rotation for the one or more activities of daily living and
providing a selection mechanism for selecting the activity of daily living to be animated.

14. (canceled)

15. The method of claim 1, further comprising:
providing a visualization of a change in a location of a center of rotation of the joint on a visualization of the model.

16. The method of claim 15, wherein the visualization of the model comprises one or more muscles visualized on the model.

17. The method of claim 16, further comprising:
providing a selection mechanism for selecting the one or more muscles to be visualized on the model.

18. The method of claim 17, further comprising:
providing a graphical visualization of the moment arm of one or more muscles selected with the selection mechanism.

19. The method of claim 17, further comprising:
providing a graphical visualization of the force-length curve through relevant ranges-of-motion for the one or more muscles selected with the selection mechanism.

20. The method of claim 1, further comprising:
receiving a selection of one or more additional implant models and placement of the models on the patient's anatomy.

21. The method of claim 20, further comprising:
providing a comparison between the originally-selected implant model and the one or more additional selected implant models.

22. The method of claim 21, further comprising:
providing a graphical visualization of the range-of-motion with respect to each of the three axes of rotation for each of the selected implant models.

23. The method of claim 22, further comprising:
providing suggestions of one or more implant positions that optimize certain parameters such as muscle moment arm, force-length curve, or minimal impingement.

24. A system for performing pre-operative planning of joint replacement surgery comprising:
a processor; and
software that, when executed on the processor, causes the system to perform the functions of:
providing a workflow, the workflow accepting as input patient-specific imaging of anatomy of a joint to be replaced using an implant from one or more imaging modalities;
deriving, from the one or more imaging modalities, a model of the joint; and
providing an application for analyzing and visualizing on the model a range-of-motion of the joint for a particular implant and placement of components of the implant on the anatomy of the joint.

25. The system of claim 24, wherein the software causes the system to perform one of the further functions of:
providing an application for analyzing and visualizing a range-of-motion of the joint required for performance of one or more activities of daily living;
providing application for analyzing and visualizing a change in a location of a center of rotation of the joint in a visualization of the model, wherein the visualization of the model comprises one or more muscles displayed on the model; and
a combination thereof.

26. (canceled)

27. (canceled)

28. A system for performing pre-operative planning of joint replacement surgery comprising:
a processor; and
software that, when executed on the processor, causes the system to perform the functions of:
providing a workflow, the workflow accepting as input patient-specific imaging of anatomy of a joint to be replaced using an implant from one or more imaging modalities;
deriving, from the one or more imaging modalities, a model of the joint; and
providing an application for analyzing and visualizing the model and a location of a center of rotation of the joint on the model;
wherein the visualization of the model comprises one or more muscles displayed on the model.
